(19) United States
(12) Patent Application Publication
(10) Pub. No.: US 2023/0234037 A1
(43) Pub. Date: Jul. 27, 2023

(54) MOLYBDENUM-BASED CATALYSTS FOR CARBON DIOXIDE CONVERSION

(71) Applicant: Air Company Holdings, Inc., Brooklyn, NY (US)

(72) Inventors: Stafford W. Sheehan, Tiverton, RI (US); Chi Chen, Shrewsbury, MA (US)

(21) Appl. No.: 17/923,758

(22) PCT Filed: May 5, 2021
(86) PCT No.: PCT/US21/30785

§ 371 Date: Nov. 7, 2022

Related U.S. Application Data
(60) Provisional application No. 63/114,779, filed on Nov. 17, 2020.
(60) Provisional application No. 63/021,989, filed on May 8, 2020.

(51) Int. Cl. B01J 23/89; C07C 29/153; B01J 27/02; B01J 35/10; B01J 37/08; B01J 21/04; B01J 21/18

(52) U.S. Cl. CPC B01J 23/8993; C07C 29/153; B01J 27/02; B01J 35/1023; B01J 35/1047; B01J 37/08; B01J 35/1042; B01J 21/04; B01J 21/185; B01J 2523/10; C07C 2523/883; C07C 2523/882; C07C 2523/881; C07C 2523/89

(57) ABSTRACT

The present invention provides a catalyst, comprising molybdenum; one or more first elements selected from a Group V, VI, VII, VIII, IX, X, and XI metal (e.g., silver, cobalt, nickel, copper, rhodium, ruthenium, iridium, palladium, niobium, and manganese); one or more second elements selected from sulfur, carbon, oxygen, phosphorus, nitrogen, and selenium; and optionally, one or more Group IA metals, wherein the molybdenum is present in an amount of 10-50 wt. % of the total amount of the one or more first elements, the molybdenum, the one or more second elements, and the Group IA metal, and methods of using said catalyst in the production of ethanol from carbon dioxide.

### RELATED APPLICATIONS

This application claims the benefit of priority to US Provisional Patent Application No. 63/021,989, filed May 8, 2020, and U.S. Provisional Patent Application No. 63/114,779, filed Nov. 17, 2020. Each of these applications is incorporated herein by reference.

### FIELD OF THE INVENTION

The present invention relates to the field of heterogeneous catalysts, specifically for catalysts that convert hydrogen gas and carbon dioxide into other materials.

### BACKGROUND OF THE INVENTION

As carbon dioxide concentrations in the atmosphere increase, it is becoming advantageous from social welfare, human health, and energy security perspectives to develop technologies that remove carbon dioxide from the air. Carbon dioxide conversion technologies have the added benefit of producing commodity chemicals on-site, anywhere on the globe, with no cost or hazard risk of transportation when coupled with air capture of CO₂. The need for removing CO₂ from the air is coupled with an increasing global utilization of renewable electricity generation methods, such as solar photovoltaics and wind turbines. Techniques like these use intermittent energy sources, such as the sun, which sets in the evening and rises in the morning, and wind, which blows intermittently. Thus, the supply of electricity from these sources to electrical grids surges at some points, and is low at others. This presents an opportunity for technologies that can intermittently utilize electricity to produce desired products on-site.

Of the available technologies to produce chemicals from carbon dioxide, hydrogenation of carbon dioxide or carbon monoxide using renewably-derived hydrogen gas from a water electrolyzer is capable of being powered completely by renewable (solar, wind, hydroelectric, etc.) electricity. A method such as this converts a carbon-based feedstock (carbon dioxide or carbon monoxide) and water into hydrocarbon chemicals using an external energy source; this is similar to the fundamental photosynthetic processes enabling life on our planet. For example, plants use photosynthesis to convert carbon dioxide, water, and solar energy into chemical energy by creating sugars and other complex hydrocarbons. This effectively stores the energy from the sun in the chemical bonds of a carbon-based compound. This process has been supporting the Earth's ecosystem and balancing carbon dioxide concentration in our atmosphere for billions of years.

In the last century, human beings have harnessed byproducts of photosynthesis, such as fossil fuels, to provide the energy required for modern life. This has released millions of tons of carbon dioxide into the Earth's atmosphere that had been previously sequestered into the fossil fuels by photosynthesis over the course of millions of years. Scientific evidence points to this rapid increase in carbon dioxide concentration in the atmosphere from anthropogenic sources to be potentially catastrophic to global climate. The development of carbon-negative processes that mimic natural ones to sequester carbon dioxide are, therefore, critical to the future of the planet, and it is an object of the present application to disclose one such invention.

One of the major hurdles toward carbon dioxide sequestration is the effective utilization and catalytic transformation of carbon dioxide or carbon monoxide into useful chemicals. Plants achieve this via dehydrogenase enzymes, which utilize transition metals to catalyze the hydrogenation of carbon dioxide into carbon monoxide, formic acid, or a number of other building blocks for sugars. Man-made systems have attempted to copy this route, and chemical methods for carbon dioxide transformation have been known for decades. Many of these, however, have energy requirements unrealistic for any large-scale deployment.

In recent years, electrochemical methods such as water electrolysis have shown promise to reduce these energy requirements to practical levels. Advances in electrochemical methods enable three such options for carbon dioxide sequestration in chemicals powered by electricity that can be sourced in a low-carbon manner: (1) electrolytic carbon dioxide reduction for one-step production of chemicals directly from carbon dioxide, (2) combined electrolysis of water to form hydrogen and oxygen gas, with subsequent hydrogenation of carbon dioxide using hydrogen gas from the electrolyzer in a high pressure, high temperature reactor in a two-step process, and (3) electrolytic carbon dioxide reduction to an intermediate that can be combined with electrochemically-derived hydrogen in a high pressure, high temperature reactor. The former process requires significant development and an improved understanding of fundamental electrocatalytic processes for carbon dioxide reduction to reach commercial viability. Specific to the production of alcohols like ethanol, integrated chemical processes require traditionally fossil-fuel based components (such as methane), with few exceptions for production of alcohols (ethanol, methanol, propanols, butanols) for any feasible further use.

In any of these processes, a crucial component is the catalyst that converts the CO₂ and hydrogen gas or hydrogen equivalents. Catalysts for CO₂ conversion, specifically, face a major challenge in that CO₂ requires a substantial amount of energy to transform into other compounds. This makes stability and activity a key challenge for industrial catalysts for CO₂ conversion. Prior to the present disclosure, because of the lack of stable catalysts for this process, no commercial chemical process was known that converts carbon dioxide into alcohols without a separate step in a chemical process that converts CO₂ to CO or CH₄ (as in the Sabatier process).

Specific to CO₂ hydrogenation, several catalysts have been demonstrated in academic literature, but none have transitioned to industrial use due to either high cost, or poor stability. Ni-based catalysts are primarily used to hydrogenate CO₂ to CH₄. Co, Fe, Ru, Ir and Rh are also catalysts for these processes, and for higher order hydrocarbon formation. Several combinations of these elements in bimetallic and trimetallic catalysts have also been demonstrated. For the formation of alcohols, catalysts comprised of Rh, Pd, Cu, Zn, Co, or Ni, supported on alumina or carbon have also been studied.

Focusing on the low-cost metals listed above that are suitable for large-scale commercial deployment (i.e. not Ru, Ir, and Rh), none have yet been demonstrated as a commercial catalyst for the hydrogenation of CO₂ to alcohols. This is primarily because they have not shown the stability that is required to scale up these systems, because they all decay into less active materials while on-stream in a reactor.

### SUMMARY OF THE INVENTION

In certain aspects, the present disclosure provides catalysts, comprising:

molybdenum;

one or more first elements selected from a Group V, VI, VII, VIII, IX, X, and XI metal (e.g., silver, cobalt, nickel, copper, rhodium, ruthenium, iridium, palladium, niobium, and manganese);

one or more second elements selected from sulfur, carbon, oxygen, phosphorus, nitrogen, and selenium; and

optionally, one or more Group IA metals,

wherein the molybdenum is present in an amount of 10-50 wt. % of the total amount of the one or more first elements, the molybdenum, the one or more second elements, and the Group IA metal.

In certain aspects, the present disclosure provides catalytic compositions, comprising the catalysts disclosed herein and a support.

In certain aspects, the present disclosure provides methods of preparing the catalysts or catalytic compositions disclosed herein, such as methods comprising preparing the catalyst by coprecipitation, wet impregnation, or ball milling.

In certain aspects, the present disclosure provides methods of hydrogenating CO₂ to a liquid product mixture, comprising contacting the catalysts or catalytic compositions disclosed herein with a feed mixture comprising CO₂ and a reductant gas at a reduction temperature and a reduction pressure, thereby providing the liquid product mixture.

### BRIEF DESCRIPTION OF THE DRAWINGS

FIG. 1: Graph showing CO₂ conversion and CO conversion of an exemplary NiCoMoSK catalyst over several hours, showing the catalyst is much better at CO₂ conversion to ethanol.

FIG. 2: Scanning electron micrograph of an exemplary NiCoMoSK catalyst.

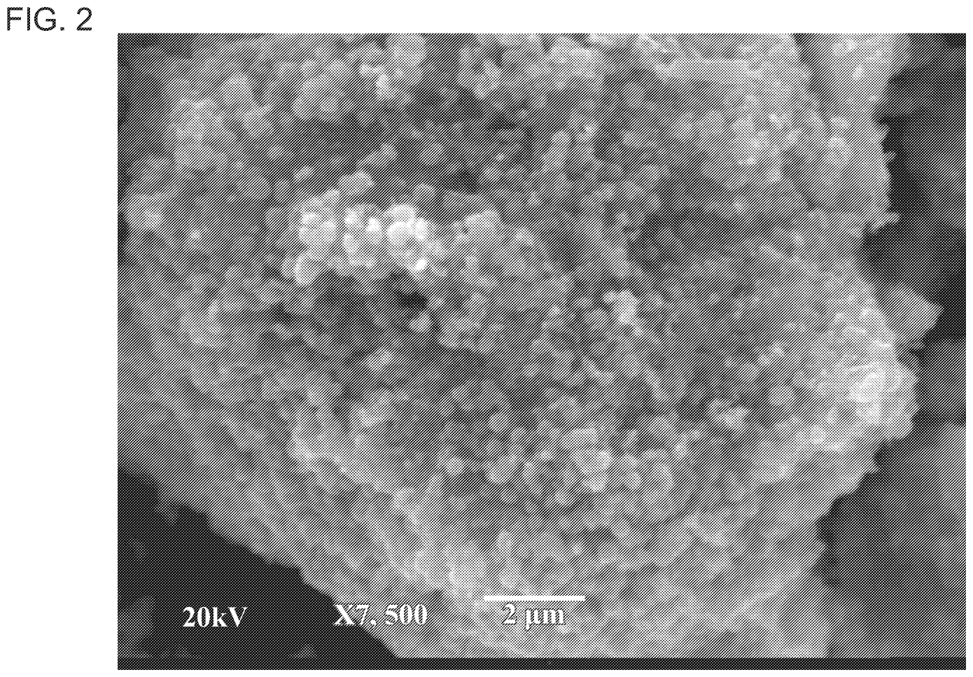

FIG. 3: Graph showing the percent of CO₂ converted per pass through a fixed-bed flow reactor for an exemplary NiCoMoSK catalyst.

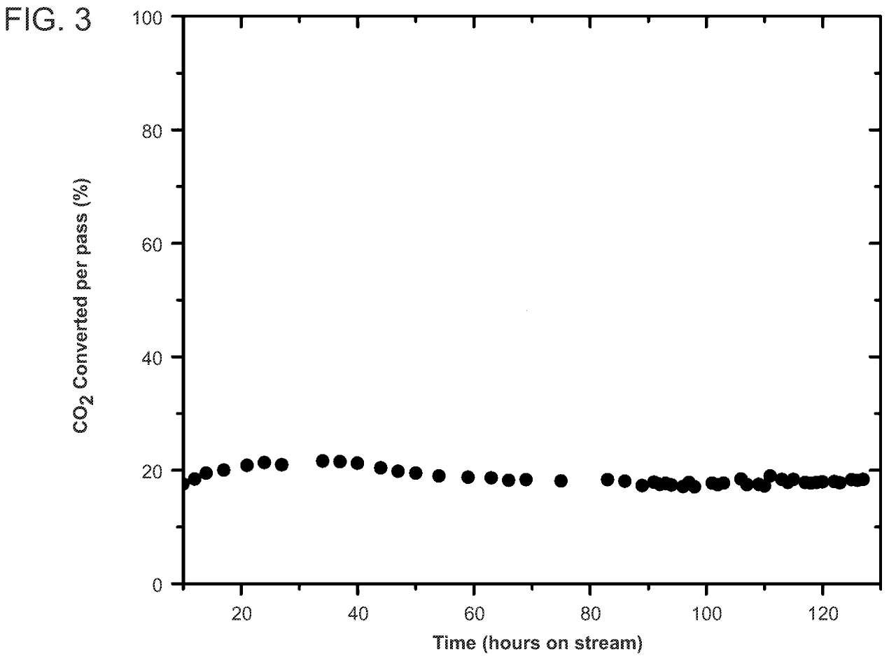

FIG. 4: Graphs showing increased ratio of carbon-containing feedstock consumption in a CO₂ and H₂ feed gas versus CO and H₂ feed gas for an exemplary NiCoMoSK catalyst.

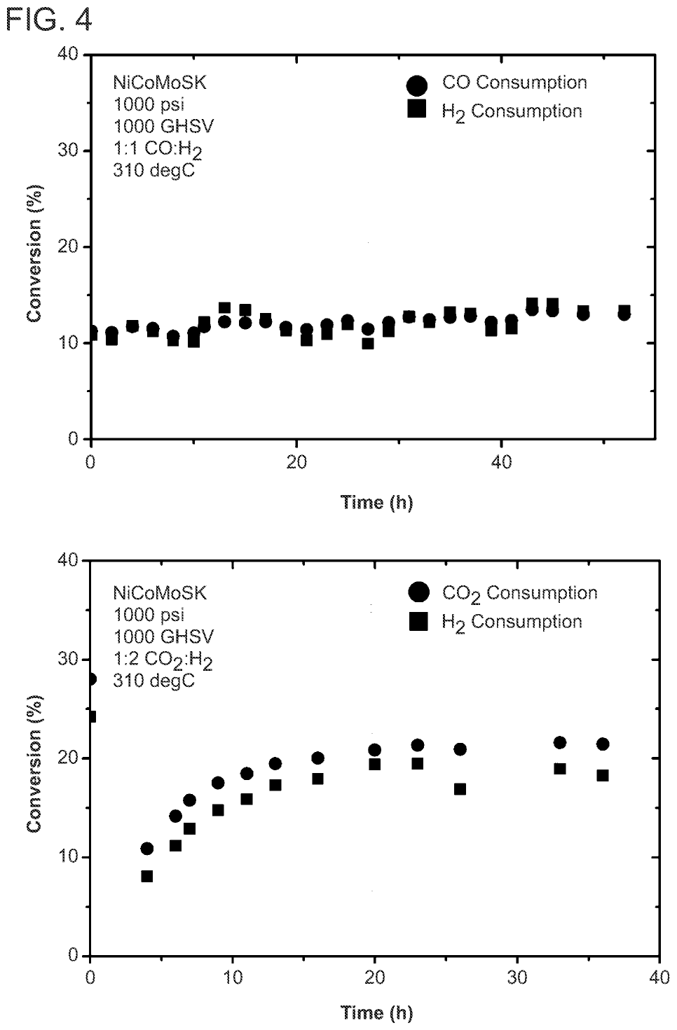

FIG. 5: Graphs showing increased ratio of H₂ consumption in a CO₂ and H₂ feed gas versus CO and H₂ feed gas for an exemplary CoMoSK catalyst.

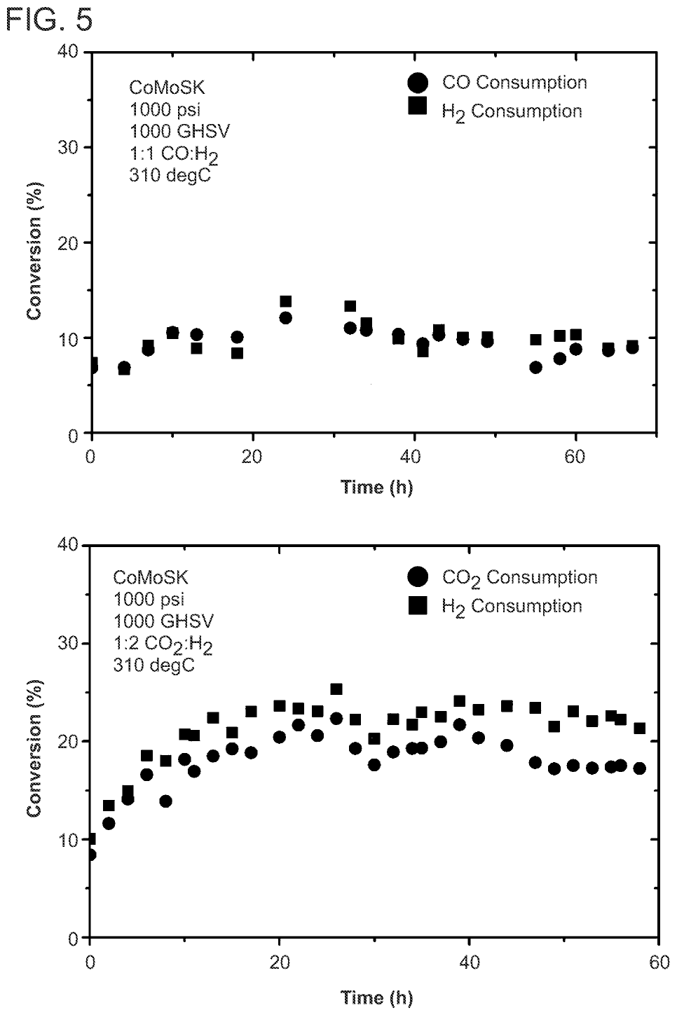

FIG. 6: Graphs showing increased ratio of carbon-containing feedstock consumption in a CO₂ and H₂ feed gas versus CO and H₂ feed gas for an exemplary CoMoSK catalyst with H₂ prereduction

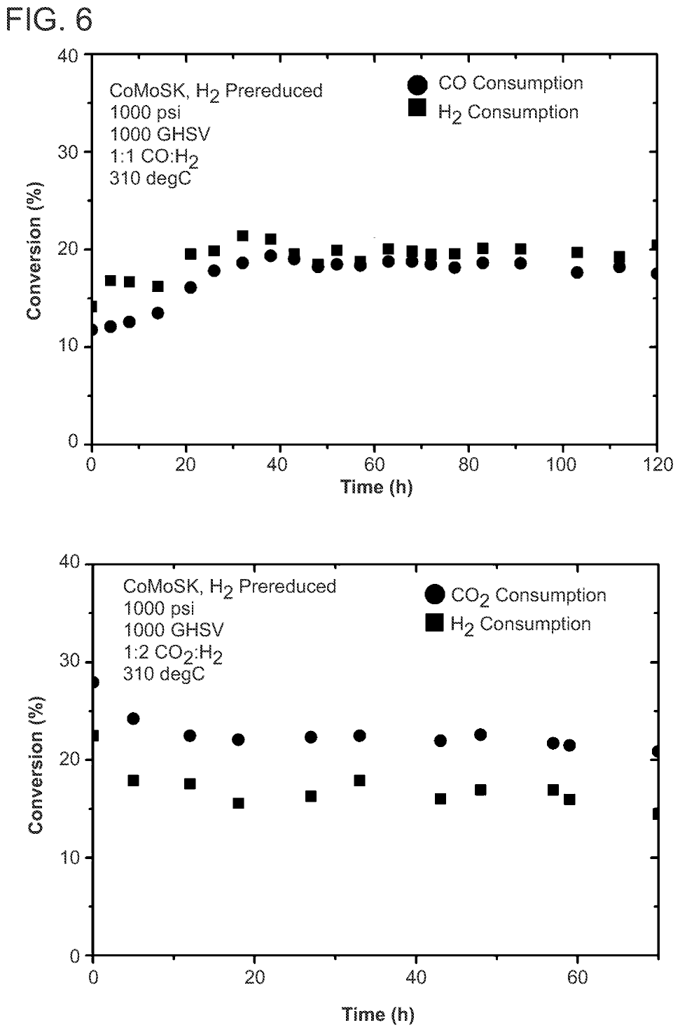

FIG. 7: Graphs showing differences in CO₂ and H₂ consumption between an exemplary CoMoSK catalyst and exemplary NiCoMoSK catalyst at 275° C.

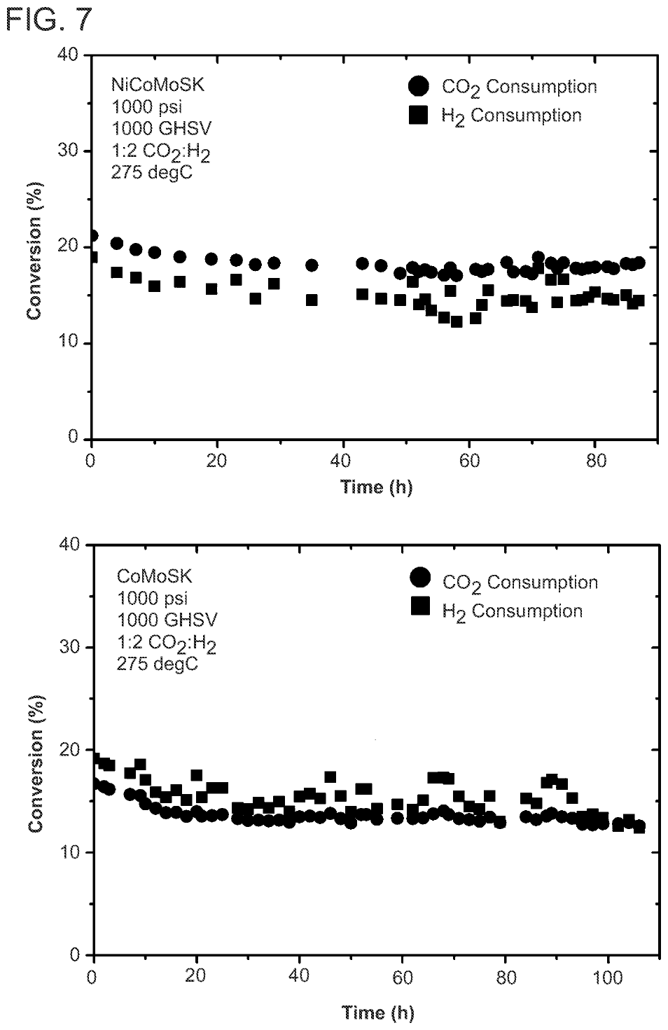

FIG. 8: Drawing of the proposed active site for an exemplary CoMoSK-type catalyst.

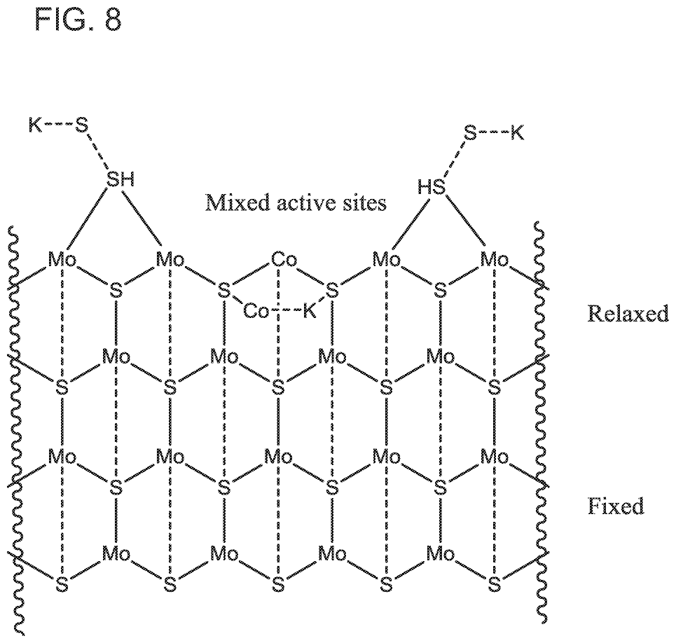

FIG. 9: Drawing of the proposed binding modes for CO₂, CO, and H₂ at the proposed active site of an exemplary CoMoSK-type catalyst.

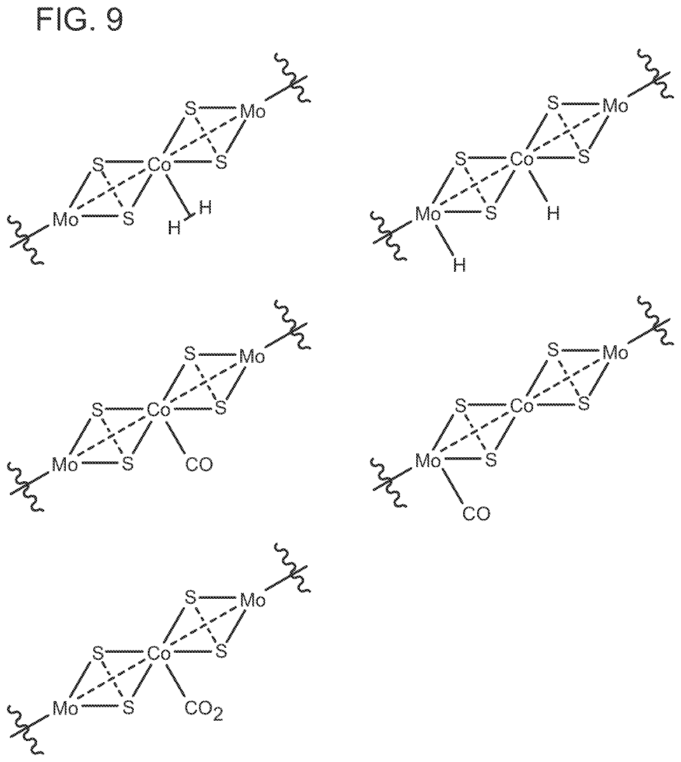

FIG. 10: Drawing of the proposed catalytic cycle for ethanol production from CO₂ and H₂ at the proposed active site of an exemplary CoMoSK-type catalyst.

### DETAILED DESCRIPTION OF THE INVENTION

The present disclosure provides Mo-based catalysts for CO₂ conversion. As further described herein, the catalysts of the present disclosure include a substantial amount of Mo as a highly active metal. Prior to the present invention, Mo-based catalysts had not been demonstrated as competent catalysts for CO₂ hydrogenation to alcohols. Among other benefits, the Mo-based catalysts of the present disclosure catalyze the production of ethanol from CO₂ feedstock at a higher rate than from CO feedstock. That is not the case with legacy CoMoSK syngas catalysts. The Mo-based catalysts of the present disclosure are also substantially more stable than catalysts that do not contain Mo.

Catalysts

In certain aspects, the present disclosure provides catalysts, comprising:

molybdenum;

one or more first elements selected from a Group V, VI, VII, VIII, IX, X, or XI metal (e.g., silver, cobalt, nickel, copper, rhodium, ruthenium, iridium, palladium, niobium, and manganese);

one or more second elements selected from sulfur, carbon, oxygen, phosphorus, nitrogen, and selenium; and

optionally, one or more Group IA metals,

wherein the molybdenum is present in an amount of 10-50 wt. % of the total amount of the one or more first elements, the molybdenum, the one or more second elements, and the Group IA metal.

In some embodiments, the molybdenum is present in an amount of 10-40 wt. %, 10-30 wt. %, or 10-20 wt. % of the total amount of the one or more first elements, the molybdenum, the one or more second elements, and the one or more Group IA metals.

In some embodiments, the molybdenum is present in an amount of 20-50 wt. %, 30-40 wt. % preferably 30-50 wt. % or, more preferably, 40-50 wt. % of the total amount of the one or more first elements, the molybdenum, the one or more second elements, and the one or more Group IA metals.

In some embodiments, the catalyst comprises one or more first elements selected from a Group VIII, IX, X, or XI metal. In some embodiments, the catalyst comprises one or more first elements selected from a Group VIII metal. In some embodiments, the catalyst comprises one or more first elements selected from a Group IX metal. In some embodiments, the catalyst comprises one or more first elements selected from a Group X metal. In some embodiments, the catalyst comprises one or more first elements selected from a Group XI metal.

In some embodiments, the one or more first elements comprise cobalt. In some embodiments, the one or more first elements comprise nickel. In some embodiments, the one or more first elements comprise silver. In some embodiments, the one or more first elements comprise copper. In some embodiments, the one or more first elements comprise niobium. In some embodiments, the one or more first elements comprise manganese.

In some embodiments, the catalyst comprises the one or more first elements at a molar ratio of about 0.15 to about 2 relative to the molybdenum. In some embodiments, the catalyst comprises the one or more first elements at a molar ratio of about 0.15 to about 1.5 relative to the molybdenum. In some embodiments, the catalyst comprises the one or more first elements at a molar ratio of about 0.15 to about 1 relative to the molybdenum. In some embodiments, the catalyst comprises the one or more first elements at a molar ratio of about 0.15 to about 0.75 relative to the molybdenum. In some embodiments, the catalyst comprises the one or more first elements at a molar ratio of about 0.15 to about 0.5 relative to the molybdenum. In some embodiments, the catalyst comprises the one or more first elements at a molar ratio of about 0.15 to about 0.25 relative to the molybdenum.

In some embodiments, the one or more first elements comprise cobalt. In some embodiments, the one or more first elements consist of cobalt. In some embodiments, the cobalt is present at a molar ratio of about 0.15 to about 2 relative to the molybdenum. In some embodiments, the cobalt is present at a molar ratio of about 0.29 relative to the molybdenum. In some embodiments, the cobalt is present at a molar ratio of about 0.2 relative to the molybdenum. In some embodiments, the cobalt is present at a molar ratio of about 0.4 relative to the molybdenum.

In some embodiments, the one or more first elements comprise nickel. In some embodiments, the one or more first elements consist of nickel. In some embodiments, the nickel is present at a molar ratio of about 0.15 to about 2 relative to the molybdenum. In some embodiments, the nickel is present at a molar ratio of about 0.36 relative to the molybdenum. In some embodiments, the nickel is present at a molar ratio of about 0.25 relative to the molybdenum. In some embodiments, the nickel is present at a molar ratio of about 0.5 relative to the molybdenum.

In some embodiments, the one or more first elements comprise silver. In some embodiments, the one or more first elements consist of silver. In some embodiments, the silver is present at a molar ratio of about 0.15 to about 2 relative to the molybdenum. In some embodiments, the silver is present at a molar ratio of about 1 relative to the molybdenum. In some embodiments, the silver is present at a molar ratio of 1.25 relative to the molybdenum. In some embodiments, the silver is present at a molar ratio of 0.75 relative to the molybdenum.

In some embodiments, the one or more first elements comprise niobium. In some embodiments, the one or more first elements consist of niobium. In some embodiments, the niobium is present at a molar ratio of about 0.05 to about 1 relative to the molybdenum. In some embodiments, the niobium is present at a molar ratio of about 0.2 relative to the molybdenum. In some embodiments, the niobium is present at a molar ratio of about 0.3 relative to the molybdenum. In some embodiments, the niobium is present at a molar ratio of about 0.1 relative to the molybdenum.

In some embodiments, the catalyst comprises the one or more Group IA metals at a molar ratio from about 0.10 to about 0.50 relative to molybdenum. In some embodiments, the catalyst comprises the one or more Group IA metals at a molar ratio from about 0.20 to about 0.50 relative to molybdenum. In some embodiments, the catalyst comprises the one or more Group IA metals at a molar ratio from about 0.30 to about 0.50 relative to molybdenum. In some embodiments, the catalyst comprises the one or more Group IA metals at a molar ratio from about 0.40 to about 0.50 relative to molybdenum. In some embodiments, the catalyst comprises the one or more Group IA metals at a molar ratio is about 0.44 relative to molybdenum. In some embodiments, the catalyst comprises potassium at a molar ratio is about 0.44 relative to molybdenum.

In some embodiments, the catalyst comprises one or more Group IA metals. In some embodiments, the one or more Group IA metals comprise potassium, sodium or cesium. In some embodiments, the one or more Group IA metals consist of potassium, sodium or cesium. In some embodiments, the one or more Group IA metals comprise potassium. In some embodiments, the one or more Group IA metals comprise sodium. In some embodiments, the one or more Group IA metals comprise cesium. In some embodiments, the one or more Group IA metals consist of potassium. In some embodiments, the one or more Group IA metals consist of sodium. In some embodiments, the one or more Group IA metals consist of cesium.

In certain embodiments, the one or more Group IA metals comprise or consist of sodium or cesium. In the catalysts of the present disclosure, substituting sodium or cesium for potassium does not substantially affect the catalytic activity, and both sodium and cesium have been found to provide the same stability potassium provides. This is a contrast with known syngas catalysts, where the choice of potassium, sodium or cesium greatly affects activity.

In some embodiments, the catalyst comprises the one or more second elements at a molar ratio from about 0.3 to about 3.25 relative to molybdenum. In some embodiments, the catalyst comprises the one or more second elements at a molar ratio from about 3 to about 3.25 relative to molybdenum. In some embodiments, the catalyst comprises the one or more second elements at a molar ratio from about 2.5 to about 3.25 relative to molybdenum. In some embodiments, the catalyst comprises the one or more second elements at a molar ratio from about 0.33 to about 3 relative to molybdenum. In some embodiments, the catalyst comprises the one or more second elements at a molar ratio from about 0.4 to about 2.5 relative to molybdenum. In some embodiments, the catalyst comprises the one or more second elements at a molar ratio from about 0.5 to about 2 relative to molybdenum. In some embodiments, the catalyst comprises the one or more second elements at a molar ratio from about 0.66 to about 1.5 relative to molybdenum.

In some embodiments, the catalyst comprises one or more second elements selected from sulfur, oxygen, selenium, or phosphorus, e.g. as a sulfide, oxide, selenide, or phosphide ion.

In some embodiments, the one or more second elements comprise sulfur. In some embodiments, the one or more second elements comprise carbon. In some embodiments, the one or more second elements comprise consist of sulfur. In some embodiments, the one or more second elements comprise phosphorus. In some embodiments, the one or more second elements comprise consist of carbon. In some embodiments, the one or more second elements comprise consist of oxygen. In some embodiments, the one or more second elements comprise consist of phosphorous. In some embodiments, the one or more second elements comprise consist of nitrogen. In some embodiments, the one or more second elements comprise consist of selenium.

In some embodiments, the sulfur is present at a molar ratio of about 3 relative to molybdenum. In some embodiments, the sulfur is present at a molar ratio of about 3.25 relative to molybdenum. In some embodiments, the sulfur is present in a molar ratio of about 2.5 relative to molybdenum. In some embodiments, the sulfur is present in a molar ratio of about 2 relative to molybdenum. In some embodiments, the carbon is present at a molar ratio of about 2.5 relative to molybdenum. In some embodiments, the carbon is present at a molar ratio of about 2 relative to molybdenum. In some embodiments, the carbon is present at a molar ratio of about 1.5 relative to molybdenum. In some embodiments, the carbon is present at a molar ratio of about 1 relative to molybdenum. In some embodiments, the carbon is present at a molar ratio of about 0.5 relative to molybdenum. In some embodiments, sulfur and carbon are both present. In some embodiments, the sulfur is present at a molar ratio of about 1 relative to molybdenum and carbon is present at a molar ratio of about 1 relative to molybdenum. In some embodiments, the carbon is present as a ‘sulfide-derived carbide’, wherein it was derived from a corresponding sulfide. In some embodiments, the nitrogen is present in a molar ratio of about 2 relative to molybdenum. In some embodiments, the nitrogen is present in a molar ratio of about 1 relative to molybdenum.

In some embodiments, the catalyst comprises silver, molybdenum, sulfur, and the Group IA metal (e.g., potassium). In some such embodiments, the molar ratios of the components are as described above. In some embodiments, the catalyst comprises: molybdenum; silver at a molar ratio of about 1 relative to the molybdenum; sulfur at a molar ratio of about 3 relative to the molybdenum; and the one or more Group IA metals (e.g., potassium) at a molar ratio of about 0.4 relative to the molybdenum.

In some embodiments, the catalyst comprises niobium, cobalt, molybdenum, sulfur, and a Group IA metal. In some embodiments, the molar ratios of the components are as described above. In some embodiments, the catalyst comprises: molybdenum; niobium at a molar ratio of about 0.12 relative to the molybdenum; cobalt at a molar ratio of about 0.60 relative to the molybdenum; sulfur at a molar ratio of about 3 relative to the molybdenum; and the Group IA at a molar ratio of about 0.44 relative to the molybdenum.

In some embodiments, the catalyst comprises nickel, cobalt, molybdenum, sulfur, and Group IA metal. In some such embodiments, the molar ratios of the components are as described above. In some embodiments, the catalyst comprises: molybdenum; nickel at a molar ratio of about 0.36 relative to the molybdenum; cobalt at a molar ratio of about 0.29 relative to the molybdenum; sulfur at a molar ratio of about 3.25 relative to the molybdenum; and the Group IA at a molar ratio of about 0.44 relative to the molybdenum.

In some embodiments, the catalyst comprises silver, cobalt, molybdenum, sulfur, and Group IA metal. In some such embodiments, the molar ratios of the components are as described above. In some embodiments, the catalyst comprises: molybdenum; silver at a molar ratio of about 0.4 relative to the molybdenum; cobalt at a molar ratio of about 0.4 relative to the molybdenum; sulfur at a molar ratio of about 3 relative to the molybdenum; and

the Group IA at a molar ratio of about 0.4 relative to the molybdenum.

In some embodiments, the catalyst comprises Co, Mo, C, and an alkali metal. In some embodiments, the catalyst comprises Ni, Co, Mo, S, and an alkali metal. In some embodiments, the catalyst comprises Ag, Mo, S, and an alkali metal. In some embodiments, the catalyst comprises Co, Mn, Mo, S, and an alkali metal. In some embodiments, the catalyst comprises Co, Nb, Mo, S, and an alkali metal.

In some embodiments, the catalyst comprises Co, Mo, and C. In some embodiments, the catalyst comprises Ni, Co, Mo, and S. In some embodiments, the catalyst comprises Ag, Mo, and S. In some embodiments, the catalyst comprises Co, Mn, Mo, and S. In some embodiments, the catalyst comprises Co, Nb, Mo, and S.

In some embodiments, the one or more second elements are present in an amount of greater than 20 wt. % of the total amount of the one or more first elements, the molybdenum, the one or more second elements, and the one or more Group IA metals. In some embodiments, sulfur is present in an amount of greater than 20 wt. % of the total amount of the one or more first elements, the molybdenum, the one or more second elements, and the one or more Group IA metals. In some embodiments, carbon is present in an amount of greater than 20 wt. % of the total amount of the one or more first elements, the molybdenum, the one or more second elements, and the one or more Group IA metals.

In certain embodiments, the elemental composition of the catalyst is CoMoCA, NiCoMoSA, AgMoSA, AgCoMoSA, AgNiMoSA, CoMnMoSA, CoNbMoCA, CoNbMoSCA or CoNbMoSA, wherein A is an alkali metal and further wherein the relative amounts of the elemental components are as described above.

In certain embodiments, the elemental composition of the catalyst is CoMoC, NiCoMoS, AgMoS, AgCoMoS, AgNiMoS, CoMnMoS, CoNbMoC, CoNbMoSC or CoNbMoS, wherein the relative amounts of the elemental components are as described above.

In some embodiments, the catalyst is selected from one of the following exemplary catalysts: CoMoC, CoMoSC, CoMoCK, CoMoSCK, NiCoMoSK, AgMoSK, CoMnMoSK, CoNbMoSK, NiCoMoCK, AgMoCK, CoMnMoCK, CoNbMoSCK, CoNbMoCK, CuMoC, CoWMoC and BiMoSK, wherein the relative amounts of the elemental components are as described above. In certain such embodiments, the catalyst is Co_{(0.6)}MoC_{(1.6)}, CO_{(0.6)}MoC_{(1.6)}K_{(0.4)}, Ni_{(0.36)}Co_{(0.29)}MoS_{(3.23)}K_{(0.44)}, AgMoS₍₃₎K_{(0.4)}, Co_{(0.6)}Mn_{(0.12)}MoS₍₃₎K_{(0.4)}, Co_{(0.6)}Nb_{(0.12)}MoS_{(3.25)}K_{(0.4)}, or Ni_{(0.36)}Co_{(0.29)}MoC₍₂₎K_{(0.44)}.

Catalytic Compositions

In certain aspects, the present disclosure provides catalytic compositions, comprising one or more of the catalysts disclosed herein and a support. The support may be any suitable material that can serve as a catalyst support.

In some embodiments, the support comprises one or more materials selected from an oxide, nitride, fluoride, or silicate of an element selected from aluminum, silicon, titanium, zirconium, cerium, magnesium, yttrium, lanthanum, zinc, and tin. In some preferred embodiments, the support comprises γ-alumina. In some embodiments, the support is an aluminum oxide. In some embodiments, the support is selected from, but not limited to, Al₂O₃, ZrO₂, SnO₂, SiO₂, ZnO, and TiO₂.

In some embodiments, the support comprises one or more carbon-based materials. In some embodiments, the carbon-based material is selected from activated carbon, carbon nanotubes, graphene and graphene oxide.

In some embodiments, the support is a mesoporous material. In some embodiments, the support has a mesopore volume from about 0.01 to about 3.0 cc/g.

In some preferred embodiments, the support has surface area from about 10 m²/g to about 1000 m²/g. In some embodiments, the catalytic composition is in a form of particles having an average size from about 20 nm to about 5 μm. In some embodiments, the catalytic composition is in a form of particles having an average size from about 50 nm to about 1 μm.

In some embodiments, the catalytic composition comprises from about 5 wt. % to about 70 wt. % of the catalyst. In some embodiments, the catalytic composition comprises from about 20 wt. % to about 70 wt. % of the catalyst. In some embodiments, the catalytic composition comprises from about 30 wt. % to about 70 wt. % of the catalyst.

In some embodiments, the support is a high surface area scaffold. In some embodiments, the support comprises mesoporous silica. In some embodiments, the support comprises carbon allotropes.

In some embodiments, the catalyst is a nanoparticle catalyst. In some embodiments, the particle sizes of the catalyst on the surface of the scaffold are 100-500 nm. In some embodiments, the particles not subjected to agglomeration are 100-500 nm in particle size.

Methods of Preparation

The catalysts and catalytic compositions of the present disclosure may be prepared by any suitable method. In certain aspects, the present disclosure provides methods for preparing the catalysts or the catalytic compositions disclosed herein, comprising preparing the catalyst by coprecipitation, wet impregnation, or ball milling.

In some embodiments, the method comprises the following steps: providing a first solution comprising a source of the one or more second elements, and combining the first solution with a molybdenum source, thereby providing a first reaction mixture; heating the first reaction mixture to a first temperature for a first period of time: providing a second solution comprising an acid, and adding a support to the second solution, thereby providing a first suspension; heating the first suspension to a second temperature for a second period of time; providing a third solution comprising a source of the one or more first elements, and adding the first reaction mixture and the third solution to the first suspension, thereby providing the second reaction mixture; heating the second reaction mixture to a third temperature for a third period of time; and isolating a solid material from the second reaction mixture.

In some embodiments, the method comprises the following steps: providing a first solution comprising a molybdenum source, a source of the one or more first elements and a source of the one or more second elements in water, and adding a support to thereby provide a first suspension; heating the first suspension to a first temperature for a first period of time; and isolating a solid material from the first suspension.

In some embodiments, the method comprises the following steps: mixing a molybdenum source and a support in a mill jar to provide a first mixture; ball milling the first mixture for between 2 hours to 2 weeks to thereby provide a first precipitate; filtering the first precipitate and heating to a first temperature to provide a ball milled molybdenum source; mixing the ball milled molybdenum source with a source of the one or more first elements and a source of the one or more second elements to provide a second mixture; and isolating a solid material from the second mixture.

In some embodiments, wherein the one or more second elements comprise carbon, the method comprises the following steps: providing an oxide catalyst precursor; and carburizing the oxide catalyst precursor with a carburization gas mixture at a carburization temperature for a carburization period of time. The carburization gas mixture may comprise any suitable gas mixture, for example methane and hydrogen, or carbon monoxide and hydrogen. In preferred embodiments, the carburization gas mixture comprises methane and hydrogen. The oxide catalyst precursor, if available commercially, may be purchased, or may be prepared by any suitable method, including by the methods disclosed herein. In certain further embodiments, providing the oxide catalyst precursor comprises providing a mixture comprising a source of the one or more first elements, a molybdenum source, and an acid (e.g., citric acid); combining the mixture with a slurry comprising a support and water, thereby providing a first suspension; heating the first suspension to a first temperature for a first period of time; isolating a solid material from the first suspension; heating the solid material at a second temperature for a second period of time, thereby providing an oxide.

In some embodiments, the method comprises the following steps: providing a mixture comprising a source of the one or more first elements, a molybdenum source, and an acid (e.g., citric acid); combining the mixture with a slurry comprising a support and water, thereby providing a first suspension; heating the first suspension to a first temperature for a first period of time; isolating a solid material from the first suspension; heating the solid material at a second temperature for a second period of time.

In some embodiments, the method further comprises combining the solid material with a source of the one or more Group IA metals. In some embodiments, the method further comprises pressing the solid material into pellets. In some embodiments, the method further comprises pressing the solid material into pellets prior to introduction into a flow reactor.

Methods of Hydrogenation

In certain aspects, the present disclosure provides methods of hydrogenating CO₂ to a liquid product mixture, comprising contacting the catalysts of catalytic compositions disclosed herein with a feed mixture comprising CO₂ and a reductant gas at a reduction temperature and a reduction pressure, thereby providing the liquid product mixture.

In some embodiments, the reductant gas is H₂. In some embodiments, the reductant gas is a hydrocarbon, such as CH₄, ethane, propane, or butane. In preferred embodiments, the hydrocarbon is CH₄. In certain such embodiments, the CH₄ is a component of a gas mixture that also comprises other hydrocarbons, such as ethane, propane, or butane. For example, the gas mixture used to supply CH₄ may be (or may be derived from) flare gas, waste gas, natural gas, or the like.

In some embodiments, the reduction temperature is from about 100 to about 600° C. In some embodiments, the reduction temperature is from about 275 to about 350° C. In some embodiments, the reduction temperature is about 275° C. In some embodiments, the reduction temperature is about 310° C.

In some embodiments, the reduction pressure is from about 250 to about 3000 psi. In some embodiments, the reduction pressure is from about 900 to about 1100 psi. In some embodiments, the reduction pressure is about 1000 psi.

In some embodiments, the molar ratio of reductant gas:CO₂ in the feed mixture is about 10:1 to about 1:10. In some embodiments, the molar ratio of reductant gas:CO₂ in the feed mixture is about 5:1 to about 0.5:1. In some embodiments, the ratio of reductant gas:CO₂ in the feed mixture is about 3:1 to about 1:1. In some embodiments, the ratio of reductant gas:CO₂ in the feed mixture is about 2:1.

In some embodiments, the liquid product mixture comprises methanol, ethanol, and n-propanol. In some embodiments, the amount of ethanol is at least 10 wt. % of the total amount of liquid product mixture. In some embodiments, the molar ratio of ethanol to the total amount of methanol and n-propanol in the liquid product mixture is from about 1:5 to about 1:10. In some embodiments, the amount of formic acid in the liquid product mixture is less than 10 ppm. In some embodiments, the amount of isopropanol in the liquid product mixture is less than 10 ppm.

In some embodiments, the method comprises contacting the catalyst with the feed mixture for at least 168 hours. In some embodiments, the method comprises contacting the catalyst with the feed mixture for at least 96 hours. In some embodiments, the method comprises contacting the catalyst with the feed mixture for at least 24 hours.

In some embodiments, the reaction temperature is between about 100° C., and about 400° C. In some embodiments, a higher temperature gives superior conversion for CO and/or CO₂ compared with lower temperature. In some embodiments, pre-reduction of the catalyst in H₂ shows a significant increase in CO₂ consumption, while H₂ consumption decreases. In some embodiments, a larger fraction of CO in the feed gas increases conversion and yield. In some embodiments, the reaction pressure is between about 300 and 3,000 psi. In some embodiments, a higher pressure gives superior conversion for CO and/or CO₂ compared with lower pressure.

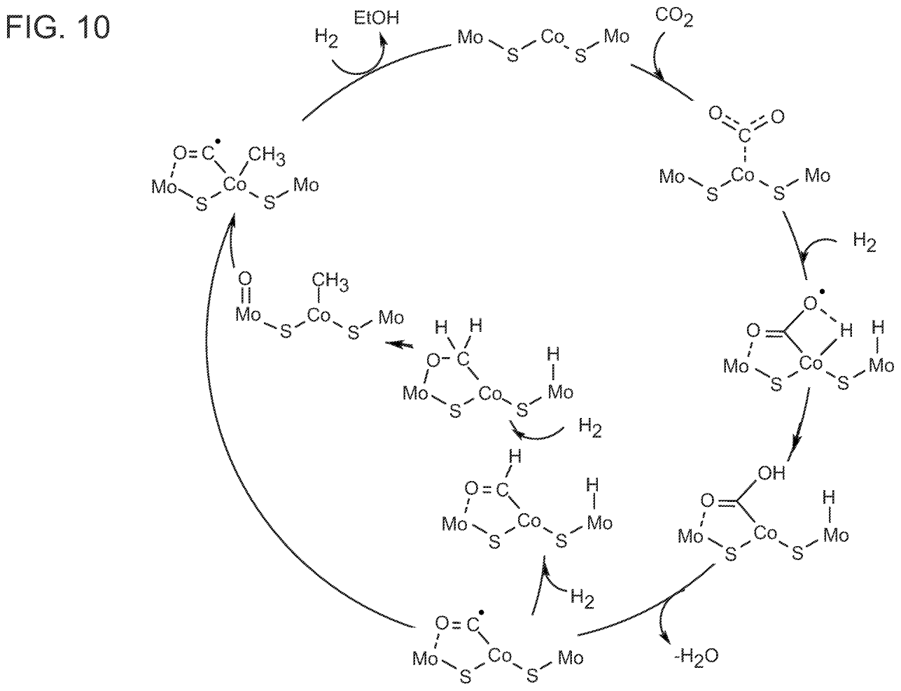

In some embodiments, the Group IA metal present in the catalyst increases the dissociative adsorption of H₂ on the surface of Mo and the first element selected from a Group V, VI, VII, VIII, IX, X, or XI metal, which are the active metals. In some embodiments, the Group IA metal donates electrons to the active metals, reducing them and promoting the oxidative addition of Ht. In some embodiments, the reduced active metals stabilize oxidative addition of H₂ into a labile dihydride complex. In some embodiments, the first element selected from a Group V, VI, VII, VIII, IX, X, or XI metal is reduced to coordinate with CO₂ wherein the adsorption of additional carbon-containing species enables chain growth to form alcohols such as ethanol or higher alcohols. In some embodiments, Mo acts as a reductant to facilitate the adsorption and activation of CO₂ by facilitating migration of oxygen and C—O bond cleavage. In some embodiments, catalysis of CO₂ and H₂ proceeds using the mechanism proposed in FIG. 10.

In some embodiments, the numbers used to describe and claim certain embodiments of the disclosure are modified in some instances by the term “about.” In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In certain embodiments, the term “about” means within 10%, 9%, 8%, 7%, 6%, 5%, 4%, 3%, 2, 1%, 0.5%, or 0.05% of a given value or range.

### EXAMPLES

The invention now being generally described, it will be more readily understood by reference to the following examples which are included merely for purposes of illustration of certain aspects and embodiments of the present invention, and are not intended to limit the invention.

### Example 1: Synthesis of Sulfide-Containing Mo-Based Catalysts by Coprecipitation

Sulfide-containing catalysts can be prepared by coprecipitating metal salts with ammonium sulfide. The precursors for the catalyst synthesis by coprecipitation are listed in Table 1; in many cases these can be substituted with a suitable comparable metal salt.

TABLE 1
Precursors for the synthesis of metal-molybdenum
sulfide catalysts.

| M1 Precursor | M2 Precursor | M-MoSA Catalyst |
|---|---|---|
| Cobalt acetate, 30 g | Nickel acetate, 30 g | NiCoMoSK |
| Silver nitrate, 30 g | None | AgMoSK |
| Silver nitrate, 15 g | Cobalt nitrate, 15 g | AgCoMoSK |
| Cobalt acetate, 30 g | Manganese sulfate, | CoMnMoSK |
| | 6 g | |
| Cobalt acetate, 30 g | Ammonium niobate | CoNbMoSK |
| | oxalate hydrate, 6 g | |
| Cobsit acetate, 30 g | Iron sulfate, 1.5 g | FeCoMoSK |

Ammonium heptamolybdate (NH₄)₆Mo₇O₂₄·4H₂O (85.5 g, 0.069 mol, 0.483 mol Mo) was added to an aqueous ammonium sulfide solution (NH₄)₂S (20 wt % in water, 0.60 L, 1.77 mol) and the mixture was heated at 60° C., for 1 hour to form a “Molybdenum solution”. An M1-Precursor (mass shown in Table 1) and an M2-Precursor (mass shown in Table 1) were dissolved in 1.1 L deionized water to form a “Metal solution”. Glacial acetic acid (675 mL) was diluted with 1.5 L deionized water to form an acetic acid solution, to which high surface area gamma alumina (29.3 g, 0.287 mol) was added to form an acidic alumina slurry, and heated to 50° C. The Metal solution and Molybdenum solution were added simultaneously to the acidic alumina slurry, which formed a black precipitate. The resulting mixture was heated at 60° C., for 1 hour and then cooled down to room temperature. The solid was filtered and dried in a fume hood for 2 days to form a highly viscous and moist catalyst paste. Solid K₂CO₃ (12.6 g, 0.091 mol) was added to the paste and mixed well with pestle and mortar. The catalyst was dried in oven at 125° C., for 3 hours and calcined at 500° C., for 1 hour with Ar flushing during the entire process, and the resulting catalyst was ground to a fine powder with a mortar and pestle.

The catalysts recited in Table 2 were prepared by coprecipitation as described above.

TABLE 2
Composition of molybdenum-based catalysts for CO₂ hydrogenation (molar ratios
of the components are indicated with respect to the amount of Mo).

| Catalyst | Scaffold | Metal 1 | Metal 2 | Mo | S | Alkali Metal (A) |
|---|---|---|---|---|---|---|
| 1A. NiCoMoSA | Alumina | Ni, 0.36 | Co, 0.29 | 1 | 3.25 | K, 0.44 |
| 2A. AgMoSA | Alumina | Ag, 1 | N/A | 1 | 3 | K, 0.4 |
| 3A. CoMnMoSA | Alumina | Co, 0.6 | Mn, 0.12 | 1 | 3 | K, 0.4 |
| 4A. CoNbMoSA | Alumina | Co, 0.6 | Nb, 0.12 | 1 | 3.25 | K, 0.4 |

Each of the above catalysts may also be prepared without the alkali metal component. Co_{(0.6)}MoS_{(3.2)} was prepared by the above method, with the omission of the addition of K₂CO₃.

TABLE 3
Composition of molybdenum-based catalysts
for CO₂ hydrogenation (molar ratios of
the components are indicated with respect
to the amount of Mo).

| Catalyst | Scaffold | Metal 1 | Metal 2 | Mo | S |
|---|---|---|---|---|---|
| 5B. CoMoS | Alumina | Co, 0.6 | N/A | 1 | 3.2 |

### Example 2: Synthesis of Carbide-Containing Mo-Based Catalysts

Carbide-containing Mo-based catalysts can be synthesized via an oxide intermediate. The oxide intermediates can be prepared by methods known in the art, such as metal coprecipitation using citric acid. Exemplary combinations of metal precursors and the resulting oxide intermediates are listed in Table 4.

TABLE 4
Metal precursors, oxide intermediates, and metal-
molybdenum carbide catalysts.

| | | Oxide | M-MoC |
|---|---|---|---|
| M1 Precursor | M2 Precursor | Intermediate | Catalyst |
| Cobalt acetate, 50 g | None | CoMoO₄ | Co_{0.6}MoC_{1.6} |
| Cobalt nitrate, 30 g | None | CoMoO₄ | Co_{0.6}MoC_{1.6} |
| Cobalt acetate 30 g | Nickel acetate 30 g | NiCoMoO₄ | NiCoMoCA |
| Silver nitrate, 30 g | None | AgMoO₃ | AgMoCA |
| Cobalt acetate, 15 g | Silver nitrate 15 g | Ag_{0.5}Co_{0.5}MoO₄ | AgCoMoCA |
| Cobalt acetate, 30 g | Manganese sulfate, | CoMn_{0.2}O₄ | CoMnMoCA |
| | 6 g | | |
| Cobalt acetate, 30 g | Ammonium niobate | CoNb_{0.2}O₄ | CoNbMoCA |
| | oxalate hydrate, 6 g | | |

Step 1: Synthesis of Oxide Intermediate by Coprecipitation of Metals with Citric Acid.

M1-Precursor (amounts shown in Table 4), M2-Precursor (amounts shown in Table 4), and ammonium heptamolybdate (NH₄)₆Mo₇O₂₄·4H₂O (85.5 g, 0.069 mol, 0.483 mol Mo) are mixed with citric acid (the amount of citric acid is equimolar to the total amount of metals in solution). The resulting mixture is completely dissolved in a slurry of gamma alumina (29.3 grams) in distilled water (1.5 L). The resulting mixture is heated at 80-90° C., for 2 hours, then dried at 120° C., overnight to remove water. The dried material is ground to a powder with a mortar and pestle, and then calcined at 550° C., for 3 hours to produce a solid powder.

Several different mass ratios of M1-Precursor to M2-Precursor can be employed to form an oxide intermediate with an optimal metal ratio. Note that, although a specific method is provided in the present example, the oxide intermediate may be prepared by any suitable method, including but not limited to, coprecipitation, ball milling, wet impregnation, and others.

### Step 2: Carburization of the Oxide Intermediate.

The oxide intermediate and the support precursor (6-8 g) are placed in a quartz sample boat, which is then placed in a quartz tube inside an STF1200 tube furnace. The system is first purged with N₂ and then subjected to a flow of 20 vol % CH₄/H₂ (50 mL/min) with a temperature programmed ramp (first heat to 280° C., at 5° C./min, then heat to 750° C., with ramp of 0.5° C./min, then hold at 750° C., for 2 hours). The sample is cooled down to 280° C., in the flow of 20 vol % CH₄/H₂, and then the sample is further cooled down in N₂ flow to room temperature. The sample is then exposed to a flow of 1 vol % O₂/N₂ for at least 2 hours to passivate the sample before removal from the oven.

Co_{(0.6)}MoC_{(1.6)}, was prepared by the above method.

For alkali-modified carbide catalysts an incipient wetness impregnation is applied using an aqueous solution of potassium carbonate sprayed onto the carbide catalyst. The impregnated samples are then aged for 1 hour, dried in N₂ at room temperature for 12-16 hours, heated in flowing N₂ with a ramp of 5° C./min to 450° C., then calcined in flowing N₂ at 450° C., for 2 hours. Alternatively, the alkali-modified carbide catalysts can be produced by dry milling of the carbide catalyst with the alkali carbonate salt.

### Addition of Further Elements.

Addition of multiple elements can be achieved by sequential impregnation steps with intermediate drying.

### Alternative Step 2: Carburization of the Sulfide Intermediate.

Alternatively, a metal molybdenum sulfide prepared by a process similar to that described in Example 1 may be subjected to the same carburization process described above for the oxide intermediate. This results in a sulfur-derived carbide. CoMoCK was prepared by this method.

### Example 3: Synthesis of Catalysts by Wet Impregnation

Wet impregnation (a.k.a. incipient wetness) synthesis: 40 grams of gamma alumina (surface area ˜185 m²/g, pore volume 0.43 cc/g) is contacted with a solution of M1-Precursor, M2-Precursor, and water, wherein the metal-containing liquid is adsorbed into the alumina by capillary action for a set period of time, typically 24 h. The sample is dried in an oven under air at 120° C., for 12 hours. The impregnated, dried sample is then ground to a powder with a mortar and pestle, heated to 550° C., for 3 h at a heating rate of 2° C./min, and calcined at 550° C., for 3 h.

### Example 4: Synthesis of Catalysts by Mechanical Activation

Mechanical activation synthesis: 50 g of molybdenum sulfide or 30 g of molybdenum carbide mixed with 20 g of gamma alumina is loaded in a 0.4 L mill jar filled ⅔ of the volume with 6.5 mm size of cylindrical grinding media, the grinding media possessing a total mass of 825 g. The mill jar is placed in a roller equipped with a % horsepower motor and the ball milling process is conducted with 200 rpm of rolling speed for different durations, between 2 hours and two weeks.

Nickel sulfide and cobalt sulfide are purchased commercially or prepared by coprecipitating 25 ml of 1.2 M aqueous solution of cobalt or nickel nitrate with 11 ml of 20% aqueous solution of ammonium disulfide. The black precipitate is filtered and heated with a heating rate of 2° C./min to 120° C. MoS₂ (2 g), cobalt sulfide (0.5 g), nickel sulfide (0.5 g) and K₂CO₃ (0.35 g) are mixed in a mortar with pestle, then ball milled to create a NiCoMoSK on alumina catalyst.

### Example 5: Synthesis of Ni_{(0.36)}Co_{(0.29)}MoS_{(3.25)}K_{(0.44)}

(NH₄)₆Mo₇O₂₄·4H₂O (85.5 g, 0.069 mol, 0.483 mol Mo) was added to (NH₄)₂S (20 wt % in water, 0.60 L, 1.77 mol) and the mixture was heated at 60° C., for 1 hour to form a Mo solution. The Mo solution was kept warm to prevent precipitation. Co(OAc)₂·4H₂O (30.0 g) and Ni(OAc)₂·4H₂O (30.0 g) was dissolved in 1.1 L DI water to form a Co solution. Acetic acid (675 mL) was dissolved in DI water 1.5 L and Al₂O₃ (29.3 g, 0.287 mol) was added and the and the mixture was heated to 50° C., to form an acetic acid solution. The Co solution and the Mo solution were added simultaneously into acetic acid solution, and the resulting mixture was heated at 60° C., for 1 hour and then cooled down to room temperature. The solid was filtered and dried in a fume hood for 2 days. K₂CO₃ (12.6 g, 0.091 mol) was added and mixed well with pestle and mortar. The catalyst was dried in an oven at 125° C., for 3 hours and calcined at 500° C., for 1 hour while Ar was flushing during the entire process. Elemental analysis confirmed the composition Ni_{(0.36)}Co_{(0.21)}MoS_{(3.25)}K_{(0.44)}.

### Example 6: Synthesis of CoMoC

Ammonium heptamolybdate (NH₄)₆Mo₇O₂₄·4H₂O (85.5 g, 0.069 mol, 0.483 mol Mo) was added to an aqueous ammonium sulfide solution (NH₄)₂S (20 wt % in water, 0.60 L, 1.77 mol) and the mixture was heated at 60° C., for 1 hour to form a “Molybdenum solution”. 60 g of cobalt acetate (Co(OAc)₂·4H₂O) was dissolved in 1.1 L deionized water to form a “Metal solution”. Glacial acetic acid (675 mL) was diluted with 1.5 L deionized water to form an acetic acid solution, to which high surface area gamma alumina (29.3 g, 0.287 mol) was added to form an acidic alumina slurry, and heated to 50° C. The Metal solution and Molybdenum solution were added simultaneously to the acidic alumina slurry, which formed a black precipitate. The resulting mixture was heated at 60° C., for 1 hour and then cooled down to room temperature. The solid was filtered and dried in a fume hood for 2 days to form a highly viscous and moist catalyst paste. Solid K₂CO₃ (12.6 g, 0.091 mol) was added to the paste and mixed well with pestle and mortar. The catalyst was dried in oven at 125° C., for 3 hours and calcined at 500° C., for 1 hour with Ar flushing during the entire process, and the resulting catalyst was ground to a fine powder with a mortar and pestle.

The sulfide intermediate was placed in a quartz sample boat, which was then placed in a quartz tube inside an STF1200 tube furnace. The system was first purged with N₂ and then subjected to a flow of 20 vol % CH₄/H₂ (50 mL/min) with a temperature programmed ramp (first heat to 280° C., at 5° C./min, then heat to 750° C., with ramp of 0.5° C./min, then hold at 750° C., for 2 hours). The sample was cooled down to 280° C., in the flow of 20 vol % CH₄/H₂, and then the sample is further cooled down in N₂ flow to room temperature. The sample was then exposed to a flow of 1 vol % O₂/N₂ for at least 2 hours to passivate the sample before removal from the oven.

### Example 7: Catalytic Reduction of CO₂ to Ethanol

For catalyst screening experiments, the Mo-based catalyst was loaded into a 600 mL continuously stirred tank reactor. The catalyst was optionally activated with H₂ prior to the start of the run. To activate the catalyst, the reactor was flushed with H₂ gas prior to being filled to 300 psi of H₂ for catalyst activation. Catalyst activation occurred at 300 psi, where the reactor was heated at 300° C., for 1.0 hour, then cooled down to 25° C., with a heating ramp rate of 6° C./min and cooling ramp rate of around −6° C./min. The reactor was vented, then flushed with 250 psi of CO₂. The reactor was filled with CO₂ to 250 psi and 500 psi of H₂ leading to a total pressure at 750 psi. The reactor was then heated to 275° C., for 15 hours prior to cooling and product collection. For product collection, the reactor was vented and disassembled to recover liquid at the bottom of the reactor. The liquid was washed and filtered to remove excess catalyst. The liquid was analyzed by nuclear magnetic resonance (NMR) to determine ethanol content to assess whether or not the catalyst was capable of producing ethanol. Copper-zinc on alumina catalysts that produce methanol from CO₂ and H₂, but little to no ethanol, were used as a standard for control experiments. Exemplary yields of ethanol in the CO₂ reduction reaction in the presence of Mo-based catalysts are listed in Table 5.

TABLE 5
Ethanol yields in CO₂ reduction in the presence
of Mo-based catalysts.

| | CuZnO₃ | Ni_{0.36}Co_{0.29} | | Ag_{0.4}Co_{0.4} |
|---|---|---|---|---|
| Catalyst | (Control) | MoS_{3.25}K_{0.44} | AgMoS₃K_{0.4} | MoS₃K_{0.4} |
| Ethanol | 0 mg | 15.6 mg | 13.6 mg | 27.3 mg |
| Yield | | | | |

For ethanol production using the catalysts of the disclosure a tubular fixed bed flow reactor was used. The optimal reactor temperature was between 275° C., and 350° C., but may vary between 200° C., and 450° C. A half-inch diameter, three foot long vertical tubular reactor was loaded with 5 mL of a mixture of catalyst powder and inert alumina. The feed ratio of gases was 2:1 H₂:CO₂, but can vary from 10:1 H₂:CO₂ to 1:10 H₂:CO₂. The gas hourly space velocity (GHSV) was 1000 h⁻¹, but can vary from 500 h⁻¹ to 20,000 h⁻¹. In some cases, gases may be recycled from the reactor back into the inlet. The pressure of the reactor was 1000 psi, however the pressure may vary from 750 psi to 3000 psi. There are generally no requirements for catalyst conditioning in these reaction systems, however, some catalysts may require heating to 300° C., under 100 psi of H₂ gas for 24 hours. Once H₂ and CO₂ gases began flowing and the reaction started, it took approximately 12 hours for the system to stabilize into a steady state where ethanol production leveled off and was no longer increasing or decreasing.

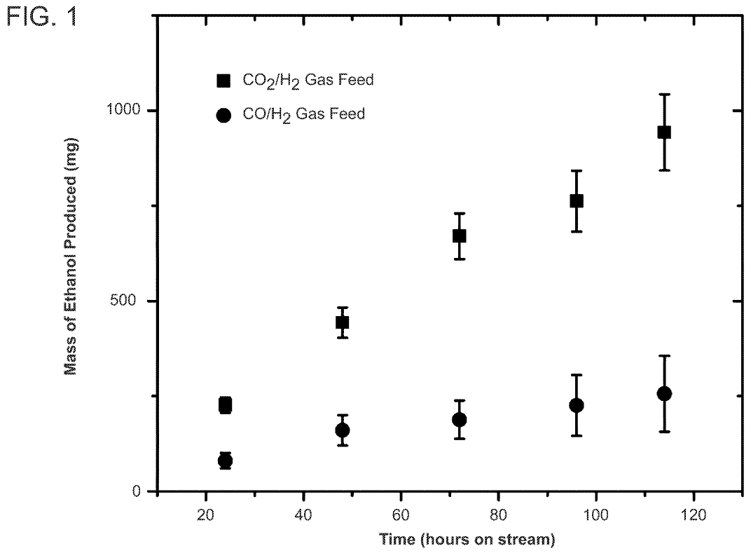

One unexpected aspect of exemplary Mo-based catalysts of the disclosure is that these catalysts afford higher ethanol production with CO₂ as a feedstock rather than CO. This is not the case with the legacy CoMoSK syngas catalysts. FIG. 1 shows the rate of ethanol production when exemplary catalysts were exposed to 2:1 H₂:CO₂ and 1:1 H₂:CO syngas, clearly showing poorer performance for the syngas. Optimal process conditions, feed gas components, and feed gas ratios may change depending on the catalyst. For example, methanol was a major byproduct of the Ni_{0.36}Co_{0.29}MoS_{3.25}K_{0.44} catalyst that was exacerbated when the reaction was performed at temperatures <300° C. Performance of Ni_{0.36}Co_{0.29}MoS_{3.25}K_{0.44} catalyst at two different temperatures is shown in Table 6.

TABLE 6
Temperature dependence of product output
during CO₂ reduction in the presence of
Ni_{0.36}Co_{0.29}MoS_{3.25}K_{0.44} catalyst.

| | Temperature: | Temperature: |
|---|---|---|
| Conditions | 275° C. | 310° C. |
| Time onstream | 96 hours | 24 hours |
| Recycle | No | No |
| Pressure | 1000 psi | 1000 psi |
| H₂:CO₂ molar ratio | 2:1 | 2:1 |
| CO₂ Conversion | 18% | 22% |
| CO Yield | 75% | 59% |
| CH₄ Yield | 1% | 10% |
| Methanol Produced (g/day) | 0.944 | 0.508 |
| Ethanol Produced (g/day) | 0.181 | 0.226 |

Stability is a key differentiator for this catalyst. It is more stable than the other ethanol producing catalysts from CO₂ in the literature. Time on stream for this catalyst totals over 3,000 hours and is tolerant of on/off cycles.

### Example 8: CO₂ Reduction in the Presence of Ni_{0.36}Co_{0.29}MoS_{3.25}K_{0.44}

CO₂ reduction in the presence of Ni_{0.36}Co_{0.29}MoS_{3.25}K_{0.44} was performed over a course of 5 days under the following conditions:

Catalyst loading 5 g;

2:1 H₂:CO₂ ratio:

GHSV was 1000 h⁻¹;

Temperature range 275-310° C.;

Pressure 1000 psi.

Composition of the liquid product fraction at different time points during the course the reaction are shown in Table 7.

TABLE 7
Composition of the liquid product fraction in CO₂ reduction
in the presence of Ni_{0.36}Co_{0.29}MoS_{3.25}K_{0.44}.

| | | | | Acetic | Formic | | |
|---|---|---|---|---|---|---|---|
| Time, h | Amount | Ethanol | Methanol | acid | acid | Acetone | Propanol |
| 24 | mmol | 4.916 | 15.884 | 0.043 | 0.000 | 0.000 | 1.577 |
| | g | 0.226 | 0.508 | 0.003 | 0.000 | 0.000 | 0.095 |
| 48 | mmol | 4.721 | 19.211 | 0.029 | 0.000 | 0.000 | 1.221 |
| | g | 0.217 | 0.615 | 0.002 | 0.000 | 0.000 | 0.073 |
| 72 | mmol | 4.925 | 29.669 | 0.034 | 0.000 | 0.000 | 1.024 |
| | g | 0.227 | 0.949 | 0.002 | 0.000 | 0.000 | 0.061 |
| 96 | mmol | 4.173 | 28.707 | 0.023 | 0.000 | 0.000 | 0.701 |
| | g | 0.192 | 0.919 | 0.001 | 0.000 | 0.000 | 0.042 |
| 120 | mmol | 3.926 | 29.495 | 0.015 | 0.000 | 0.000 | 0.605 |
| | g | 0.181 | 0.944 | 0.001 | 0.000 | 0.000 | 0.036 |

### Example 9: Catalytic Reduction of CO₂ to Alcohols Using CH₄ as a Reductant

For catalyst screening experiments, the Mo-based catalyst is loaded into a 600 mL continuously stirred tank reactor. The catalyst is optionally activated with H₂ prior to the start of the run. To activate the catalyst, the reactor is flushed with H₂ gas prior to being filled to 300 psi of H₂ for catalyst activation. Catalyst activation occurs at a pressure of at least 100 psi, where the reactor is heated at 300° C., for 1.0 hour, then cooled down to 25° C., with a heating ramp rate of 6° C./min and cooling ramp rate of around −6° C./min. The reactor is vented, then flushed with 250 psi of CO₂. The reactor is filled with CO₂ to 250 psi and 500 psi of CH₄ leading to a total pressure at 750 psi. The reactor is then heated to 250° C., for 15 hours prior to cooling and product collection. For product collection, the reactor is vented and disassembled to recover liquid at the bottom of the reactor. The liquid is washed and filtered to remove excess catalyst. The liquid is analyzed by gas chromatography (GC) to determine methanol, ethanol, n-propanol, and higher alcohol content to assess whether the catalyst is capable of producing alcohols using CO₂ and CH₄.

For alcohol production using the catalysts disclosed in this specification, a tubular fixed bed flow reactor is typically used, but other reactor types may also be used. For the example of a tubular fixed bed flow reactor, the optimal reactor temperature is between 200° C., and 300° C., but may vary between 100° C., and 450° C. A half-inch diameter, three foot long vertical tubular reactor is loaded with 5 mL of a mixture of catalyst powder and, optionally, inert alumina to even out temperature differences within the reactor during exothermal operation. The feed ratio of gases is 2:1 CH₄:CO₂, but can vary from 10:1 CH₄:CO₂ to 1:10 CH₄:CO₂, optionally with the presence of other carbonaceous gases such as CO. The gas hourly space velocity (GHSV) in the present example is 1000 h⁻¹, but can vary from 100 h⁻¹ to 75,000 h⁻¹. In some cases, gases that are unreacted in their first pass through the reactor may be recycled from the reactor back into the inlet. The pressure of the reactor is 1000 psi, however the pressure may vary from 500 psi to 5000 psi. There are sometimes no requirements for catalyst conditioning in these reaction systems, however, some catalysts may require heating to temperatures as high as 400° C., under at least 100 psi of H₂, CO, or CH₄ gas for up to 24 hours. Once CH₄ and CO₂ gases begin flowing and the reaction starts, it takes approximately 12 hours for the system to stabilize into a steady state where alcohol production levels off and is no longer increasing or decreasing.

### Example 10: Pressure, Temperature, and Feed Gas Composition Effects on CO₂ and H₂ Consumption

CO₂ and CO reduction in the presence of CoMoSK and NiCoMoSK was performed for varied time periods under the following conditions:

Catalyst loading 5 g;

With and without prereduction under H₂ at 310° C.;

1:1 H₂:CO ratio and 2:1 H₂:CO₂ ratio;

GHSV was 1000 h⁻¹:

Temperature range 275-310° C.:

Pressure range 750-1000 psi.

High temperature (310° C.) resulted in better consumption for both CO/CO₂ and H₂ compared with lower temperature runs (275° C.). CoMoSK at 310° C., resulted in 22% CO₂ consumption and 16% H₂ consumption while at 275° C., resulted in 16% CO₂ consumption and 11% H₂ consumption. CoNiMoSK at 310° C., resulted in 20% CO₂ consumption and 18% H₂ consumption while at 275° C., it resulted in 18% CO₂ consumption and 15% H₂ consumption. Operations with prereduction of catalyst under flowing H₂ showed a significant increase in CO₂ consumption and decrease in H₂ consumption. CoMoSK runs at 310° C., with prereduction resulted in 22% CO₂ consumption and 16% H₂ consumption while runs at 310° C., without prereduction resulted in 16% CO₂ consumption and 22% H₂ consumption. CoMoSK runs at 275° C., with prereduction resulted in 16% CO₂ consumption and 11% consumption while runs at 275° C., without prereduction resulted in 14% CO₂ consumption and 16% H₂ consumption. When using a 1:1 CO:H₂ feedstock, CoMoSK with prereduction resulted in 16% CO consumption and 18% H₂ consumption while runs without prereduction resulted in 10% CO consumption and 10% H₂ consumption.

Lower pressure generally lowers consumption. For example, CoMoSK runs at 310° C. 1000 psi resulted in 22% CO₂ consumption and 16% H₂ consumption while runs at 310° C., and 750 psi resulted in 19% CO₂ consumption and 14% H₂ consumption. Prereduction can significantly improve the reactivity of CO while only mildly improving CO₂ runs. For example. CoMoSK runs at 310° C., with CO₂ and pre-reduced in H₂ resulted in 22% CO₂ consumption and 16% H₂ consumption while runs at 310° C., with CO resulted in 16% CO consumption and 18% H₂ consumption. While CoMoSK runs at 310° C., without prereduction with CO₂ resulted in 16% CO₂ consumption and 22% consumption while runs at 275° C., without prereduction resulted in 10% CO₂ consumption and 10% H₂ consumption. NiCoMoSK runs at 310° C., with catalyst prereduction resulted in 20% CO₂ consumption and 18% H₂ consumption while runs at 275° C., without prereduction resulted in 11% CO₂ consumption and 11% H₂ consumption.

With the inclusion of Ni, CO₂ consumption was increased while H₂ consumption dropped slightly. CoMoSK runs at 310° C., without prereduction resulted in 16% CO₂ consumption and 22% H₂ consumption while runs at 310° C., with NiCoMoSK without prereduction resulted in 20% CO₂ consumption and 18% H₂ consumption. CoMoSK runs at 275° C., without prereduction resulted in 14% CO₂ consumption and 16% H₂ consumption while runs at 275° C., with NiCoMoSK without prereduction resulted in 18% CO₂ consumption and 15% H₂ consumption.

### Example 11: Comparison of CoMnMoSK and CoNbMoSK

CoMnSMoSK and CoNbMoSK were synthesized as detailed previously and CO₂ reduction was performed under the following conditions:

Catalyst loading 5 g;

2:1 H₂:CO₂ ratio;

GHSV was 1000 h⁻¹;

Temperature was 275° C.;

Pressure was 1000 psi.

The conversion of CO₂ stabilized at 16% for the CoMnMoSK and 18% for the CoNbMoSK. The CoNbMoSK had much lower CH₄ selectivity (12%) compared to the CoMnMoSK (22%). The CoNbMoSK had an overall selectivity for alcohols of approximately 22% and produced a liquid with about 4.1% ethanol by weight and a 0.45 ratio of ethanol to methanol over a 233 hour test.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A catalyst, comprising:
molybdenum;
one or more first elements selected from a Group V, VI, VII, VIII, IX, X, and XI metal (e.g., silver, cobalt, nickel, copper, rhodium, ruthenium, iridium, palladium, niobium, and manganese);
one or more second elements selected from sulfur, carbon, oxygen, phosphorus, nitrogen, and selenium; and
optionally, one or more Group IA metals,
wherein the molybdenum is present in an amount of 10-50 wt. % of the total amount of the one or more first elements, the molybdenum, the one or more second elements, and the Group IA metal.

2. The catalyst of claim 1, wherein the one or more first elements comprise silver, cobalt, nickel, copper, rhodium, ruthenium, iridium, palladium, niobium, or manganese.

3. The catalyst of claim 1 or 2, wherein the one or more first elements comprise cobalt.

4. The catalyst of any one of the preceding claims, wherein the one or more first elements comprise nickel.

5. The catalyst of any one of the preceding claims, wherein the one or more first elements comprise silver.

6. The catalyst of any one of the preceding claims, wherein the one or more first elements comprise copper.

7. The catalyst of any one of the preceding claims, wherein the one or more first elements comprise niobium.

8. The catalyst of any one of the preceding claims, wherein the one or more first elements comprise manganese.

9. The catalyst of any one of the preceding claims, wherein the catalyst comprises the one or more first elements at a molar ratio of about 0.15 to about 2 relative to the molybdenum.

10. The catalyst of any one of the preceding claims, wherein the catalyst comprises cobalt at a molar ratio of about 0.15 to about 2 relative to the molybdenum.

11. The catalyst of any one of the preceding claims, wherein the catalyst comprises cobalt at a molar ratio of about 0.29 relative to the molybdenum.

12. The catalyst of any one of the preceding claims, wherein the catalyst comprises nickel at a molar ratio of about 0.15 to about 2 relative to the molybdenum.

13. The catalyst of any one of the preceding claims, wherein the catalyst comprises nickel at a molar ratio of about 0.36 relative to the molybdenum.

14. The catalyst of any one of the preceding claims, wherein the catalyst comprises silver at a molar ratio of about 0.15 to about 2 relative to the molybdenum.

15. The catalyst of any one of the preceding claims, wherein the catalyst comprises silver at a molar ratio of about 1 relative to the molybdenum.

16. The catalyst of any one of the preceding claims, wherein the catalyst comprises one or more Group IA metals.

17. The catalyst of claim 16, wherein the one or more Group IA metals comprise potassium.

18. The catalyst of claim 16, wherein the one or more Group IA metals comprise sodium.

19. The catalyst of claim 16, wherein the one or more Group IA metals comprise cesium.

20. The catalyst of any one of claims 16-19, wherein the catalyst comprises the one or more Group IA metals at a molar ratio from about 0.10 to about 0.50 relative to molybdenum.

21. The catalyst of claim 20, wherein the catalyst comprises the one or more Group IA metals to molybdenum at a molar ratio of about 0.44 relative to molybdenum.

22. The catalyst of claim 20 or 21, wherein the one or more Group IA metals comprise potassium.

23. The catalyst of any one of the preceding claims, wherein the catalyst comprises the one or more second elements at a molar ratio from about 0.3 to about 3.25 relative to molybdenum.

24. The catalyst of claim 23, wherein the catalyst comprises the one or more second elements at a molar ratio from about 3 to about 3.25 relative to molybdenum.

25. The catalyst of claim 23, wherein the catalyst comprises the one or more second elements at a molar ratio from about 2.5 to about 3.25 relative to molybdenum.

26. The catalyst of any one of the preceding claims, wherein the one or more second elements comprise sulfur.

27. The catalyst of any one of the preceding claims, wherein the one or more second elements comprise carbon.

28. The catalyst of claim 26, wherein the catalyst comprises sulfur at a molar ratio of about 3.25 relative to molybdenum.

29. The catalyst of claim 1, wherein the catalyst comprises silver, molybdenum, sulfur, and a Group IA metal.

30. The catalyst of claim 29, wherein the catalyst comprises:
molybdenum;
silver at a molar ratio of about 1 relative to the molybdenum;
sulfur at a molar ratio of about 3 relative to the molybdenum; and
the Group IA at a molar ratio of about 0.4 relative to the molybdenum.

31. The catalyst of claim 1, wherein the catalyst comprises nickel, cobalt, molybdenum, sulfur, and a Group IA metal.

32. The catalyst of claim 31, wherein the catalyst comprises:
molybdenum;
nickel at a molar ratio of about 0.36 relative to the molybdenum;
cobalt at a molar ratio of about 0.29 relative to the molybdenum;
sulfur at a molar ratio of about 3.25 relative to the molybdenum; and
the Group IA at a molar ratio of about 0.44 relative to the molybdenum.

33. The catalyst of claim 1, wherein the catalyst comprises niobium, cobalt, molybdenum, sulfur, and a Group IA metal.

34. The catalyst of claim 33, wherein the catalyst comprises:
niobium at a molar ratio of about 0.12 relative to the molybdenum;
cobalt at a molar ratio of about 0.6 relative to the molybdenum;
sulfur at a molar ratio of about 3.25 relative to the molybdenum; and
the Group IA at a molar ratio of about 0.4 relative to the molybdenum.

35. A catalytic composition, comprising the catalyst of any one of the preceding claims, and a support.

36. The catalytic composition of claim 35, wherein the support comprises one or more materials selected from an oxide, nitride, fluoride, or silicate of an element selected from aluminum, silicon, titanium, zirconium, cerium, magnesium, yttrium, lanthanum, zinc, and tin.

37. The catalytic composition of claim 35 or 36, wherein the support comprises γ-alumina.

38. The catalytic composition of claim 35, wherein the support comprises one or more carbon-based material.

39. The catalytic composition of claim 38, wherein the carbon-based material is selected from activated carbon, carbon nanotubes, graphene, and graphene oxide.

40. The catalytic composition of any one of claims 35-39, wherein the support is a mesoporous material.

41. The catalytic composition of claim 40, wherein the support has a mesopore volume from about 0.01 to about 3.0 cc/g.

42. The catalytic composition of any one of claims 35-41, wherein the support has surface area from about 10 m²/g to about 1000 m²/g.

43. The catalytic composition of any one of claims 35-42, wherein catalytic composition comprises from about 5 wt. % to about 70 wt. % of the catalyst.

44. The catalytic composition of any one of claims 35-43, wherein the catalytic composition is in a form of particles having an average size from about 20 nm to about 5 μm.

45. The catalytic composition of any one of claims 35-44, wherein the catalytic composition is in a form of particles having an average size from about 50 nm to about 1 μm.

46. The catalytic composition of any one of claims 35-45, wherein the catalytic composition is in a form of particles having an average size from about 100 nm to about 500 nm.

47. The catalytic composition of any one of claims 35-45, wherein the catalytic composition is in a form of particles having an average size from about 50 nm to about 300 nm.

48. A method for preparing the catalyst of any one of claims 1-34 or the catalytic composition of any one of claims 35-47, comprising preparing the catalyst by coprecipitation, wet impregnation, or ball milling.

49. The method of claim 48, comprising the following steps:
providing a first solution comprising a source of the one or more second elements, and combining the first solution with a molybdenum source, thereby providing a first reaction mixture;
heating the first reaction mixture to a first temperature for a first period of time;
providing a second solution comprising an acid, and adding a support to the second solution, thereby providing a first suspension;
heating the first suspension to a second temperature for a second period of time;
providing a third solution comprising a source of the one or more first elements, and adding the first reaction mixture and the third solution to the first suspension, thereby providing the second reaction mixture;
heating the second reaction mixture to a third temperature for a third period of time; and
isolating a solid material from the second reaction mixture.

50. The method of claim 48, comprising the following steps:
providing a first solution comprising a molybdenum source, a source of the one or more first elements and a source of the one or more second elements in water, and adding a support to thereby provide a first suspension;
heating the first suspension to a first temperature for a first period of time; and
isolating a solid material from the first suspension.

51. The method of claim 48, comprising the following steps:
mixing a molybdenum source and a support in a mill jar to provide a first mixture;
ball milling the first mixture for a time period between 2 hours to 2 weeks to thereby provide a first precipitate;
filtering the first precipitate and heating to a first temperature to provide a ball milled molybdenum source;
mixing the ball milled molybdenum source with a source of the one or more first elements and a source of the one or more second elements to provide a second mixture; and
isolating a solid material from the second mixture.

52. The method of claim 48, wherein the one or more second elements comprise carbon, comprising the following steps:
providing an oxide catalyst precursor;
carburizing the oxide catalyst precursor with a carburization gas mixture at a carburization temperature for a carburization period of time.

53. The method of claim 52, wherein the carburization gas mixture comprises methane and hydrogen.

54. The method of claim 52, wherein the carburization gas mixture comprises carbon monoxide and hydrogen.

55. The method of claim 52, wherein providing the oxide catalyst precursor comprises:
providing a mixture comprising a source of the one or more first elements, a molybdenum source, and an acid;
combining the mixture with a slurry comprising a support and water, thereby providing a first suspension;
heating the first suspension to a first temperature for a first period of time;
isolating a solid material from the first suspension;
heating the solid material at a second temperature for a second period of time, thereby providing an oxide.

56. The method of claim 48, comprising:
providing a mixture comprising a source of the one or more first elements, a molybdenum source, and an acid;
combining the mixture with a slurry comprising a support and water, thereby providing a first suspension;
heating the first suspension to a first temperature for a first period of time;
isolating a solid material from the first suspension;
heating the solid material at a second temperature for a second period of time.

57. The method of any one of claims 48-56, further comprising combining the solid material with a source of the one or more Group IA metals.

58. A method for hydrogenating CO₂, comprising contacting the catalyst of any one of claims 1-34 or the catalytic composition of any one of claims 35-47 with a feed mixture comprising CO₂ and a reductant gas at a reduction temperature and a reduction pressure, thereby providing a liquid product mixture.

59. The method of claim 58, wherein the reductant gas is H₂.

60. The method of claim 58, where the reductant gas is a hydrocarbon, such as CH₄, ethane, propane, or butane.

61. The method of claim 58, wherein the reductant gas is, or is derived from, flare gas, waste gas, or natural gas.

62. The method of claim 58, wherein the reductant gas is CH₄.

63. The method of any one of claims 58-62, wherein the reduction temperature is 100 to 600° C.

64. The method of any one of claims 58-63, wherein the reduction pressure is 500 to 3000 psi.

65. The method of any one of claims 58-64, wherein the molar ratio of reductant gas:CO₂ in the feed mixture is from about 10:1 to about 1:10.

66. The method of any one of claims 58-65, wherein the molar ratio of reductant gas:CO₂ in the feed mixture is about 5:1 to about 0.5:1.

67. The method of any one of claims 58-66, wherein the liquid product mixture comprises methanol, ethanol, and n-propanol.

68. The method of claim 67, wherein the amount of ethanol is at least 10 wt. % of the total amount of liquid product mixture.

69. The method of any one of claims 55-68, comprising contacting the catalyst with the feed mixture for 24 hours.

70. The method of claim 69, comprising contacting the catalyst with the feed mixture for 96 hours.

71. The method of claim 70, comprising contacting the catalyst with the feed mixture for 168 hours.

72. The method of any one of claims 58-71, wherein the molar ratio of ethanol to the total amount of methanol and n-propanol in the liquid product mixture is from about 1:5 to about 1:10.

73. The method of any one of claims 58-72, wherein the amount of formic acid in the liquid product mixture is less than 10 ppm.

74. The method of any one of claims 58-73, wherein the amount of isopropanol in the liquid product mixture is less than 10 ppm.

75. The method of any one of claims 58-74, further comprising reacting the catalyst or catalytic composition with the reductant gas prior to reacting with the feed mixture.
